SEQUENCE LISTING (2 sequences)

SEQ ID NO: 1 - DNA, 1501 bp, Homo sapiens
  CDS (229)..(1347)
ttcctattccaggtctctgctgctgatgaagctgtgaccaaacgcacccaacccttggcagccatctgtccctgcagccatagcccacattcccatgacctccctctgcttgttttgggaccatgtctgtacagcctctaggccccagccccggaggtgaatgccatgccatgattctggagagcaccatggcatccccagcctagctcccaatcccactttggcacgatgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccgccatcgtgcacccgctgcgactgcgccacctgcggcggccccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctgaacacacatgccattgcgctgtccgtgcccgactcccaacgcctctcgttctgggaggcttacagggtgtacacacaagaaggtgggctgggcacttggacctttgggtggcaattccagcttagcaacgcagaagagtacaaagtgtggaagcc

SEQ ID NO: 2 - protein, 372 aa, Homo sapiens [sequence omitted]

(19) United States
(12) Patent Application Publication
(10) Pub. No.: US 2002/0115102 A1
(43) Pub. Date: Aug. 22, 2002

(54) Novel protein

(76) Inventors: Diane Joan Cousens, Stevenage (GB); Steven Michael Foord, Stevenage (GB); Diane Michele Ignar, Cary, NC (US); Filippo Volpe, Stevenage (GB)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 09/768,894

(22) Filed: Jan. 24, 2001

(30) Foreign Application Priority Data
Jan. 25, 2000 (GB) GB 0001700.4

(51) Int. Cl. G01N 33/53; C07H 21/04; C12P 21/02; C12N 5/06; C07K 14/705; C07K 16/28

(52) U.S. Cl. 435/7.1; 435/69.1; 435/325; 435/320.1; 530/350; 530/388.22; 536/23.5

(57) ABSTRACT

A purinergic like polypeptide comprises

(i) the amino acid sequence of SEQ ID NO:2 or

(ii) a variant thereof which maintains a basic functionality of the polypeptide having the amino acid sequence of SEQ ID NO:2 (HIPHUM0000001).

### PRIORITY APPLICATION INFORMATION

This application claims priority to application number GB 0001700.4 filed Jan. 25, 2000.

### FIELD OF THE INVENTION

The present invention relates to purinergic receptor like polypeptides.

### BACKGROUND OF THE INVENTION

A number of purinergic receptors for extracellular ATP have been reported since 1993. These P2 purinergic receptors can be subdivided into two classes: P2X receptors (ion channels) and P2Y receptors (G-protein coupled 7-transmembrane receptors). Within the P2Y family five functional human receptors (P2Y1, 2, 4, 6 & 11) have been identified with distinct agonist specificities. In addition a number of uncloned purinergic receptors have been postulated based on specific pharmacology, the most well known of these being the P2T receptor, currently classified within the P2Y family, which is found on human platelets. Purinergic-like orphan receptors (P2Y5, 9 and 10) have also been postulated based on protein homology but to date these remain unresponsive to a wide range of purinergic ligands.

Purinergic receptors are known to be involved in neurotransmission, ADP-induced platelet shape change and aggregation, lung mucociliary clearance and relaxation of smooth muscle. Purinergic receptors have also been suggested to have roles in the cardiovascular system, gastro-intestinal (GI) tract, immune system and endocrine system.

### SUMMARY OF THE INVENTION

A novel purinergic receptor-like polypeptide, referred to herein as HIPHUM0000001 is now provided which is a screening target for the identification and development of novel pharmaceutical agents, including agonists and antagonists of the purinergic receptor-like polypeptide. HIPHUM0000001 has been found to be primarily expressed in the tonsil and adenoid, with reasonable levels of expression also shown in the ileum, thyroid, brain, colon and adipose tissue. These agents may be used in the treatment and/or prophylaxis of disorders such as acute and chronic inflammatory disease, cardiovascular diseases, disorders of the brain, disorders of the reproductive system, disorders of the GI tract, disorders of the thyroid and adipose related disorders.

Accordingly, the present invention provides an isolated purinergic receptor-like polypeptide comprising

(i) the amino acid sequence of SEQ ID NO: 2, or

(ii) a variant thereof which maintains a functionality of the polypeptide having the amino acid sequence of SEQ ID NO: 2 (HIPHUM0000001).

According to another aspect of the invention there is provided a polynucleotide encoding a polypeptide of the invention which polynucleotide comprises:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

The invention also provides:

an expression vector comprising a polynucleotide of the invention;

a host cell comprising an expression vector of the invention;

a method of producing a polypeptide of the invention which method comprises maintaining a host cell of the invention under conditions suitable for obtaining expression of the polypeptide and isolating the said polypeptide;

an antibody specific for a polypeptide of the invention;

a method for identification of a compound that stimulates a polypeptide having the amino acid sequence of SEQ ID NO: 2 (HIPHUM0000001) activity, which method comprises contacting a polypeptide of the invention with a test compound and monitoring for receptor activity in the presence of a G-protein;

a method for identification of a compound that exhibits modulating activity of a polypeptide having the amino acid sequence of SEQ ID NO: 2 (HIPHUM0000001) which method comprises contacting a polypeptide of the invention with a purinergic receptor-like agonist in the presence of a test compound and monitoring purinergic receptor-like activity in the presence of a G-protein.

a compound which stimulates or modulates a polypeptide having the amino acid sequence of SEQ ID NO: 2 (HIPHUM0000001) activity and which is identifiable by the method referred to above;

a method of treating a subject having a disorder that is responsive to stimulation or modulation of a polypeptide having the amino acid sequence of SEQ ID NO: 2 (HIPHUM0000001), which method comprises administering to said patient an effective amount of a compound of the invention; and

use of a compound that stimulates or modulates a polypeptide having the amino acid sequence of SEQ ID NO: 2 (HIPHUM0000001) activity in the manufacture of a medicament for the treatment or prophylaxis of a disorder that is responsive to stimulation or modulation of HIPHUM0000001.

### BRIEF DESCRIPTION OF THE FIGURES

FIG. 1 shows the tissue distribution of HIPHUM0000001 expression in normal human tissues.

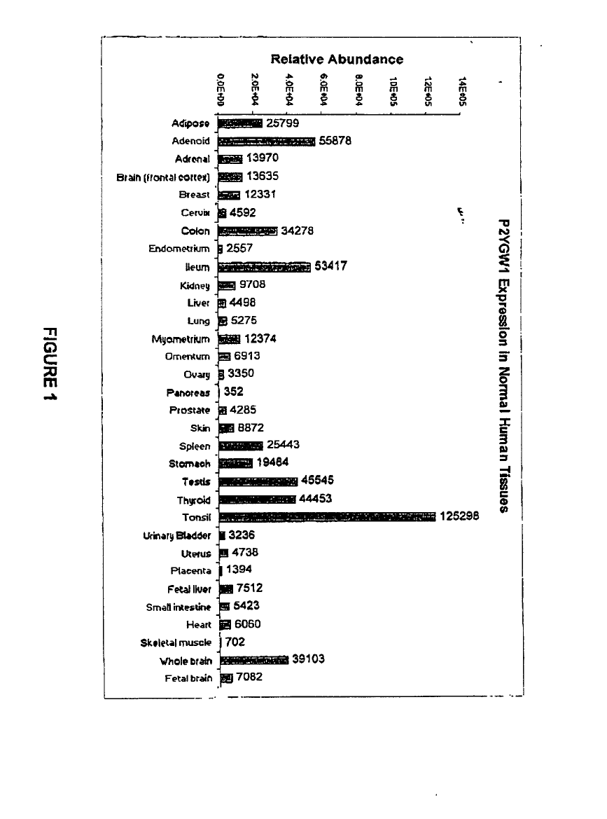

### BRIEF DESCRIPTION OF THE SEQUENCES

SEQ ID No 1 shows the DNA and amino acid sequence of human protein HIPHUM0000001 and its encoding DNA.

SEQ ID No 2 shows the amino acid sequence alone of HIPHUM0000001.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human purinergic-like receptor, referred to herein as HIPHUM0000001, and variants thereof. Sequence information for HIPHUM0000001 is provided in SEQ ID NO: 1 (nucleotide and amino acid) and in SEQ ID NO: 2. The polypeptides of the invention consist essentially of the amino acid sequence of SEQ ID NO: 2 or of a variant of that sequence.

The polypeptides are provided in isolated form. The term “isolated” is intended to convey that the polypeptide is not in its native state, insofar as it has been purified at least to some extent or has been synthetically produced, for example by recombinant methods. The polypeptide may be mixed with carriers or diluents which will not interfere with the intended purpose of the polypeptide and still be regarded as substantially isolated. The term “isolated” therefore includes the possibility of the polypeptide being in combination with other biological or non-biological material, such as cells, suspensions of cells or cell fragments, proteins, peptides, expression vectors, organic or inorganic solvents, or other materials where appropriate, but excludes the situation where the polypeptide is in a state as found in nature.

A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press (1989), the disclosure of which is included herein in its entirety by way of reference.

The term “variants” refers to polypeptides which have the same essential character or a biological functionality of HIPHUM0000001. The function of HIPHUM0000001 can be defined as follows: HIPHUM0000001 is a purinergic receptor-like. A polypeptide having the same essential character as HIPHUM0000001 couples to G protein in oocytes. Preferably a variant of the invention will be modulated by the same agonist and antagonist as HIPHUM0000001.

Agonist activity can be determined by contacting cells expressing a polypeptide of the invention with a potential agonist and by monitoring the effect of the potential agonist on the polypeptide. Antagonist activity can be determined by incubating cells expressing a polypeptide of the invention with a potential antagonist, contacting the cells with an agonist, and monitoring the inhibition of receptor activation by the agonist. Typically, polypeptides with more than about 65% identity preferably at least 80% or at least 90% and particularly preferably at least 95% at least 97% or at least 99% identity, with the amino acid sequences of SEQ ID NO: 2, are considered as variants of the proteins. Such variants may include allelic variants and the deletion, modification or addition of single amino acids or groups of amino acids within the protein sequence, as long as the polypeptide maintains a biological functionality of HIPHUM0000001.

Amino acid substitutions may be made, for example from 1, 2 or 3 to 10, 20 or 30 substitutions. The modified polypeptide generally retains activity as a purinergic receptor. Conservative substitutions may be made, for example according to the following Table. Amino acids in the same block in the second column and preferably in the same line in the third column may be substituted for each other.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates a biological functionality of HIPHUM0000001. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Fragments from which the C-terminus has been removed are preferred as the C-terminus is necessary for desensitisation to occur. Such fragments can be used to construct chimeric receptors preferably with another 7-transmembrane receptor, more preferably with another member of the P2Y family of purinergic receptors. Such fragments can also be used to raise anti-HIPHUM0000001 antibodies.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM0000001 or variants thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al. Such nucleotides can typically be isolated from tonsil, adenoid, ileum, testis, thyroid, brain, colon and adipose tissue.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO:1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO:1 is typically at least 10 fold, preferably at least 100 fold, as intense as interactions between other polynucleotides and the coding sequence of SEQ ID NO:1. The intensity of interaction may be measured, for example, by radiolabelling the probe, e.g. with ³²P. Selective hybridisation may typically be achieved using conditions of low stringency (0.03 M sodium chloride and 0.03 M sodium citrate at about 40° C.), medium stringency (for example, 0.03 M sodium chloride and 0.03 M sodium citrate at about 50° C.) or high stringency (for example, 0.03 M sodium chloride and 0.03 M sodium citrate at about 60° C.).

The coding sequence of SEQ ID No:1 may be modified by nucleotide substitutions, for example from 1, 2 or 3 to 10, 25, 50 or 100 substitutions. The polynucleotide of SEQ ID NO:1 may alternatively or additionally be modified by one or more insertions and/or deletions and/or by an extension at either or both ends. The modified polynucleotide generally encodes a polypeptide which has HIPHUM0000001 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO:1 will generally have at least 60%, at least 70%, at least 80%, at least 90%, at least 95%, at least 98% or at least 99% sequence identity to the coding sequence of SEQ ID NO:1 over a region of at least 20, preferably at least 30, for instance at least 40, at least 60, more preferably at least 100 contiguous nucleotides or most preferably over the full length of SEQ ID NO:1.

Sequence identity of polynucleotide or peptide sequences of the invention can be calculated using the CGC (University of Wisconsin) suite of programs and in particular using Clustal W. The default matrix for all protein-protein comparisons is preferably BLOSUM 62. For nucleotide sequences, the blast 2.0 program may be used. Altschul et al 1997 “GappedBLAST and PSI-BLAST”.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM0000001, or antisense sequences, may also be used in gene therapy, such as in strategies for down regulation of expression of the proteins of the invention.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO:1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al.

The invention also includes cells that have been modified to express the HIPHUM0000001 polypeptide or a variant thereof or a cell line modified in this way. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation and cell surface expression of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention.

It is also possible for the proteins of the invention to be transiently expressed in a cell line or on a membrane, such as for example in a baculovirus expression system. Such systems, which are adapted to express the proteins according to the invention, are also included within the scope of the present invention.

According to another aspect, the present invention also relates to antibodies (either polyclonal or preferably monoclonal antibodies) which have been raised by standard techniques and are specific for a polypeptide of the invention. Such antibodies could for example, be useful in purification, isolation or screening methods involving immunoprecipitation techniques and may be used as tools to further elucidate the function of HIPHUM0000001 or a variant thereof, or indeed as therapeutic agents in their own right. Antibodies may also be raised against specific epitopes of the proteins according to the invention.

An important aspect of the present invention is the use of polypeptides according to the invention in screening methods to identify compounds that may act as agonists or which may modulate HIPHUM0000001 receptor activity. Any suitable form may be used for the assay to identify a modulator of HIPHUM0000001 activity. In general terms, such screening methods may involve contacting a polypeptide of the invention with a test compound and then measuring receptor activity or may involve incubating a polypeptide of the invention with a test substance and then detecting modulation of agonist activity at the receptor. Agents which bind to the proteins of the present invention can also be identified by binding assays.

Agonist activity can be determined by contacting cells expressing a polypeptide of the invention with a substance under investigation and by monitoring the effect mediated by the polypeptide. The cells expressing the polypeptide may be in vitro or in vivo. The polypeptide of the invention may be naturally or recombinantly expressed. Preferably, in vitro cells expressing recombinant polypeptide are used. Other methods that can typically be used to monitor HIPHUM0000001 activity, involve measuring levels of or activity of GTPγS, GTPase, cAMP or inisitol triphosphate (IP3). A microphysiometer may be utilized when determining HIPHUM0000001 activity. ³⁵S-ATP binding, reporter assays, overexpression systems (including semliki forest virus (SFV), baculovirus, yeast and melanophore systems) and electrophysiological measurements of ion channel modulation may also be used to determine HIPHUM0000001 activity.

Typically receptor activity can be monitored indirectly by measuring Gᵢ-coupled readout. Gᵢ coupled readout can typically be monitored using an electrophysiological method to determine the activity of G-protein regulated Ca²⁺ or K⁺ channels or by using a fluorescent dye to measure changes in intracellular Ca²⁺ levels. Preferably control experiments are carried out in cells which do not express the polypeptide of the invention to establish whether the observed responses are resulting from activation of the polypeptide.

It is preferred that a dose-response curve is constructed by repeating the experiment using increasing concentrations of the test substance. For each agonist, it may be useful to identify the molar concentration of an agonist which produces 50% of the maximum possible response for that agonist for each protein. The more potent the agonist, the smaller will be its EC50 value. Comparison of the EC50s for different agonists will identify whether an agent is a partial agonist. Alternatively, it may be possible to compare the EC50s to establish the relative response to a protein of the invention to a number of different agonists. Variants of the invention which show the same relative response can then readily be identified by performing the same comparison.

An active test substance may be a full or partial agonist for the polypeptide. The maximal response produced by a partial agonist will be submaximal compared to the maximal response produced in response to a full agonist.

Antagonist activity can be determined by incubating cells expressing a polypeptide of the invention with a substance under investigation, contacting the cells with an agonist, and monitoring the effect of the test substance by measuring agonist activity as described above. It is preferred that a dose-response curve for the agonist is constructed first in the absence, then in the presence, of the test substance to enable the degree of antagonism and whether the antagonism observed is reversible or irreversible to be determined. It is also preferred that dose response curves are obtained in the presence of various antagonist concentrations. pA2 may be used to define the activity of an antagonist and is the negative log of the concentration of antagonist which would produce a 2-fold shift in the concentration response curve for an agonist. The ability of a test substance to potentiate the effect of an agonist for the polypeptide of the invention can be determined by similar methods. An antagonist or agonist for a variant of HIPHUM0000001 may have different values for pA2, Emax or EC50 compared to the values of that antagonist or agonist for HIPHUM0000001. However the effect of the comparative effect of the compounds on HIPHUM0000001 and the variant will be similar.

The binding of an agonist or antagonist to a polypeptide of the invention can also be determined directly. For example, a radiolabelled test substance can be incubated with a polypeptide of the invention and binding of the test substance to the polypeptide can be monitored. Typically, the radiolabelled test substance can be incubated with cell membranes containing the polypeptide until equilibrium is reached. The membranes can then be separated to a non-bound test substance and dissolved in scintillation fluid to allow the radioactive content to be determined by scintillation counting. Non-specific binding of the test substance may also be determined by repeating the experiments in the presence of a saturating concentration of a non-radioactive ligand. Preferably a binding curve is constructed by repeating the experiments with various concentrations of the test substance.

Following purinergic-receptor stimulation, cyclic AMP accumulation can be measured for example in forskolin stimulated CHO cells transformed with the HIPHUM0000001 receptor either directly, or indirectly by monitoring the expression of cotransfected reporter gene, the expression of which will be controlled by cyclic AMP response elements.

Xenopus dermal melanophores aggregate or disperse pigment in response to the activation or inhibition of G protein coupled receptors. This feature can be exploited as an assay for receptor activation or inhibition if a specific G protein coupled receptor is exogenously expressed.

HIPHUM0000001 receptor is likely to couple to G protein with consequent hydrolysis of GTP. Accumulation of a labelled GTP stable analogue can be measured utilising membrane fractions from overexpression of HIPHUMOOOOOO1 receptor either in X. laevis oocytes or cell lines such as HEK293, CHO, COS7, HeLa on exposure to agonist ligand.

G protein coupled receptors have been shown to activate MAPK signalling pathways. Cell lines overexpressing the purinergic-like receptor with MAPK reporter genes may be utilised as assays for receptor activation or inhibition. The purinergic-receptor of the invention may be heterologously expressed in modified yeast strains containing multiple reporter genes, such as FUS 1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain two further gene deletions, such as SST2 and FAR1, to potentiate the assay. Ligand activation of the heterologous receptor can be monitored using the reporter genes, for example either as cell growth in the absence of histidine or with a substrate of beta-galactosidase (lacZ).

Additional control experiments may be carried out, for example in the absence of the ligand or using known agonists or antagonists of other purinergic receptors.

Suitable test substances which can be tested in the above assays include combinatorial libraries, defined chemical entities, peptide and peptide mimetics, oligonucleotides and natural product libraries, such as display (e.g. phase display libraries) and antibody products.

Test substances may be used in an initial screen of, for example, 10 substances per reaction, and the substances of these batches which show inhibition or activation tested individually. Test substances may be used at a concentration of from 1 nM to 1000:M, preferably from 1:M to 100:M, more preferably from 1:M to 10:M. A stimulator of HIPHUM0000001 receptor activity is one that itself activates HIPHUM0000001. A modulator of HIPHUM0000001 activity is one which produces a measurable reduction or increase in the receptor activity in response to agonists in the assays described above.

Another aspect of the present invention is the use of polynucleotides encoding the HIPHUM0000001 polypeptides of the invention to identify mutations in HIPHUM0000001 genes which may be implicated in human disorders. Identification of such mutations may be used to assist in diagnosis of immune system, ileum, colon, brain, thyroid, testis disorders or susceptibility to such disorders and in assessing the physiology of such disorders.

Another aspect of the present invention is the use of the compounds that have been identified by screening techniques referred to above in the treatment or prophylaxis of disorders which are responsive to regulation of HIPHUM0000001 activity. In particular, such compounds may be used in the treatment of disorders such as acute and chronic inflammatory disease, such as asthma, chronic obstructive pulmonary disease (COPD), allergic rhinitis, hayfever, immune deficiency disorder, AIDS, rheumatoid arthritis, multiple sclerosis, leukaemia, myesthenia, gravis, graves 1 disease, systemic lupus erythematosus, inflammatory bowel disease, encephalomyelitis, psoriasis, atopic dermantitis, septic shock, stroke, ischaemia reperfusion injury, cardiovascular diseases, disorders of the brain including Alzheimer's, epilepsy, stroke, pain, disorders of the reproductive system including infertility, MED, disorders of the GI tract including constipation, diarrhea, malabsorption, polyps, ulceritive colitis, and chron's disease, disorders of the thyroid including hyperthyroidism, hypothyroidism goitre, cretinism, myxoedema, breast cancer, adipose related disorders such as hyperlipidemia, obesity, hyper and hypo-lipoproteinaemia, diabetes, and cancer.

It is to be understood that mention of these specific disorders is by way of example only and is not intended to be limiting on the scope of the invention as described.

The compounds identified according to the screening methods outlined above may be formulated with standard pharmaceutically acceptable carriers and/or excipients as is routine in the pharmaceutical art, and as fully described in Remmington's Pharmaceutical Sciences, Mack Publishing Company, Eastern Pennsylvania 17^{th} Ed. 1985, the disclosure of which is included herein of its entirety by way of reference.

The compounds may be administered by enteral or parenteral routes such as via oral, buccal, anal, pulmonary, intravenous, intra-arterial, intramuscular, intraperitoneal, topical or other appropriate administration routes. The dose of a compound to be administered may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the compound, the age, weight and conditions of the subject to be treated, the type and severity of the disease and the frequency and route of administration. Preferably, daily dosage levels are from 5 mg to 2 g.

The following Examples illustrate the invention.

### Example 1

### Measurement of Agonist and Antagonist Activity at HIPHUM0000001 using a Fluorescence Imaging Plate Reader (FLIPR)

Mammalian cells (1321N1 human astrocytoma cells) stably over-expressing HIPHUM0000001 are cultured in black wall, clear bottom, tissue culture coated 96 or 384 well plates with a volume of 100:1 cell culture medium in each well 3 days before use in a FLIPR (Fluorescence Imaging Plate Reader B Molecular Devices). Cells were incubated with 4:M FLUO B 3AM at 37° C. in 5%CO₂ for 90 mins and then washed once in Tyrodes buffer containing probenecid 3 mM. Basal fluorescence was determined prior to compound additions. The protein is activated upon the addition of an agonist. Activation results in an increase in intracellular calcium which can be measured directly in the FLIPR. For antagonist studies, compounds were preincubated with the cells for 4 minutes following dye loading and washing and fluorescence measured for 4 minutes. Agonists were then added and cell fluorescence measured for a further 1 minute.

Xenopus Oocyte Expression

Adult female Xenopus laevis (Blades Biologicals) were anaesthetised using 0.2% tricaine (3-aminobenzoic acid ethyl ester), killed and the ovaries rapidly removed. Oocytes were then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes were transfered to ND96 solution (96 mM NaCl, 2 mM KCl, lmM MgCl₂, 5 mM HEPES, 2.5 mM sodium pyruvate; pH 7.5 at 25° C.) which contained 50 μg ml⁻¹ gentamycin and stored at 18° C.

The purinergic-like receptor (in pcDNA₃, Invitrogen) was linearised and transcribed to RNA using T7 (Promega Wizard kit). m′G(5′)pp(5′)GTP capped cRNA was injected into oocytes (20-50 ng per oocyte) and whole-cell currents were recorded using two-microelectrode voltage-clamp (Geneclamp amplifier, Axon instruments Inc.) 3 to 7 days post-RNA injection. Microelectrodes had a resistance of 0.5 to 2 MΩ when filled with 3 M KCl.

1. An isolated purinergic receptor-like polypeptide comprising
(i) the amino acid sequence of SEQ ID NO: 2 or
(ii) a variant thereof which maintains a basic functionality of the polypeptide having the amino acid sequence of SEQ ID NO: 2 (HIPHUM0000001).

2. A polypeptide according to claim 1 wherein the variant 10 (ii) has at least 80% identity to the amino acid sequence of SEQ ID NO: 2.

3. A polynucleotide encoding a polypeptide according to claim 1.

4. A polynucleotide encoding a purinergic receptor-like polypeptide which polynucleotide comprises:
(a) the nucleic acid sequence of SEQ ID NO: 1 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 60% identity to a sequence as defined in (a), (b) or (c).

5. A polynucleotide according to claim 3 which is a cDNA sequence.

6. An expression vector comprising a polynucleotide sequence according to claim 3.

7. A host cell comprising an expression vector according to claim 6.

8. An antibody specific for a polypeptide according to claim 1.

9. A method for identification of a compound that stimulates a polypeptide having the amino acid sequence of SEQ ID NO: 2 (HIPHUM0000001) activity, which method comprises contacting a polypeptide according to claim 1 with a test compound and monitoring for receptor activity in the presence of a G-protein.

10. A method for identification of a compound that modulates a polypeptide having the amino acid sequence of SEQ ID NO: 2 (HIPHUM0000001) activity which method comprises contacting a polypeptide according to claim 1 with an agonist in the presence of a test compound and monitoring receptor activity in the presence of a G-protein.

11. A method according to claim 9 wherein the polypeptide is expressed in a cell.

12. A compound which stimulates or modulates a polypeptide having the amino acid sequence of SEQ ID NO: 2 (HIPHUM0000001) receptor activity and which is identifiable by a method according to claim 9.

13. A method of treating a subject having a disorder that is responsive to modulation, a polypeptide having the amino acid sequence of SEQ ID NO: 2 (HIPHUM0000001) which method comprises administering to said subject an effective amount of a compound according to claim 12.

14. A method according to claim 13 wherein the disorder is selected from an acute and chronic inflammatory disease, such as asthma, chronic obstructive pulmonary disease (COPD), allergic rhinitis, hayfever, immune deficiency disorder, AIDS, rheumatoid arthritis, multiple sclerosis, leukaemia, myesthenia, gravis, graves disease, systemic lupus erythematosus, inflammatory bowel disease, encephalomyelitis, psoriasis, atopic dermantitis, septic shock, stroke, ischaemia reperfusion injury, cardiovascular diseases, disorders of the brain including Alzheimer's, epilepsy, stroke, pain, disorders of the reproductive system including infertility, MED, disorders of the GI tract including constipation, diarrhea, malabsorption, polyps, ulceritive colitis, and chron's disease, disorders of the thyroid including hyperthyroidism, hypothyroidism goitre, cretinism, myxoedema, breast cancer, adipose related disorders such as hyperlipidemia, obesity, hyper and hypo-lipoproteinaemia, diabetes, and cancer

15. Use of a compound as defined in claim 12 in the manufacture of a medicament for treatment or prophylaxis of a disorder that is responsive to stimulation or modulation of a polypeptide having the amino acid sequence of SEQ ID NO: 2 (HIPHUM0000001).

16. A use according to claim 15 wherein the disorder is selected from an acute and chronic inflammatory disease, such as asthma, chronic obstructive pulmonary disease (COPD), allergic rhinitis, hayfever, immune deficiency disorder, AIDS, rheumatoid arthritis, multiple sclerosis, leukaemia, myesthenia, gravis, graves disease, systemic lupus erythematosus, inflammatory bowel disease, encephalomyelitis, psoriasis, atopic dermantitis, septic shock, stroke, ischaemia reperfusion injury, cardiovascular diseases, disorders of the brain including Alzheimer's, epilepsy, stroke, pain, disorders of the reproductive system including infertility, MED, disorders of the GI tract including constipation, diarrhea, malabsorption, polyps, ulceritive colitis, and chron's disease, disorders of the thyroid including hyperthyroidism, hypothyroidism goitre, cretinism, myxoedema, breast cancer, adipose related disorders such as hyperlipidemia, obesity, hyper and hypo-lipoproteinaemia, diabetes, and cancer.

17. A method of producing a polypeptide according to claim 1, which method comprises maintaining a cell line as defined in claim 7 under conditions suitable for obtaining expression of the polypeptide and isolating the said polypeptide.
